(19) United States
(12) Patent Application Publication
(10) Pub. No.: US 2015/0111303 A1
(43) Pub. Date: Apr. 23, 2015

(54) SYNTHESIS OF MOLECULE ON NANOPARTICLE SURFACE FOR STABLE DETECTION OF NITROAROMATIC EXPLOSIVES, AND SENSOR USING SAME

(71) Applicant: POSTECH ACADEMY-INDUSTRY FOUNDATION, Pohang-si (KR)

(72) Inventors: Sungjee Kim, Pohang-si (KR); Nayoun Won, Seoul (KR); Jungheon Kwag, Gimhae-si (KR); Joonhyuck Park, Pohang-si (KR); Ho Jin, Seosan-si (KR)

(21) Appl. No.: 14/399,878

(22) PCT Filed: May 2, 2013
(86) PCT No.: PCT/KR2013/003790

§ 371 Date: Nov. 7, 2014

(30) Foreign Application Priority Data
May 8, 2012 (KR) 10-2012-0048748

(51) Int. Cl. G01N 33/22

(52) U.S. Cl. CPC G01N 33/227
USPC 436/110

(57) ABSTRACT

The present invention relates to a nanoparticle-based nitroaromatic explosive sensor for detecting nitroaromatic compounds, more specifically to stably detecting explosives in an aqueous solution by introducing, on the surface of the nanoparticles, a molecule which improves the dispersion force of the nanoparticles in an aqueous solution while binding strongly therewith, and which can simultaneously bind with the nitroaromatic compounds.

### TECHNICAL FIELD

The present invention relates to a nanoparticle-based nitroaromatic explosive sensor for detecting nitroaromatic compounds, and more particularly, to stable detection of explosives in an aqueous solution by introducing, on the surface of nanoparticles, a molecule that may enhance dispersion force of the nanoparticles in an aqueous solution while being strongly bound to the nanoparticles, and also that may be coupled with nitroaromatic compounds.

### BACKGROUND ART

Typical compounds useful as explosives include nitroaromatic chemicals such as trinitrotoluene (TNT) or dinitrotoluene (DNT). A variety of methods for detecting such chemicals have been developed.

Research and development into methods of detecting chemicals contained in explosives using ion mobility spectroscopy or neutron detection is ongoing, but these methods are problematic because of relatively long detection time and high cost, compared to when using biosensors.

Furthermore, sensors using changes in absorption or fluorescence of nanoparticles are mainly being devised these days. Such sensors may be provided in the form of a simple measurement device, and are efficiently applicable as a real-time explosive sensor due to its short sensing time.

Detection of TNT using nanoparticles may be illustratively performed in a manner that employs nanoparticles, the surface of which is introduced with a molecule having a terminal primary amine group, or in such a manner that TNT anions are attracted to a positively charged amine ligand due to formation of a Meisenheimer complex between a primary amine group and TNT or due to acid-base interaction between amine and TNT. However, the nanoparticles conventionally used to detect TNT are configured such that a portion of the surface ligand bound to the nanoparticles is a thiol group and thus bonding force with the nanoparticles is comparatively weak, and the other terminal is made up of a primary amine group and thus sensitively reacts depending on changes in pH or ion intensity, thus forming a cluster of nanoparticles or deteriorating the dispersion force.

With the recent environmental problems due to nitroaromatic compounds such as TNT, there is a need for a stable sensor in a wide pH range under high ion intensity conditions in order to directly detect TNT from an environmental sample such as sea water or groundwater.

### DISCLOSURE

### Technical Problem

Accordingly, an object of the present invention is to provide a nanoparticle able to stably detect explosives in a wide pH range and at high ion intensity.

Another object of the present invention is to provide a novel compound, which enables stable explosive detection using the nanoparticle.

A further object of the present invention is to provide a method of increasing explosive detection sensitivity using the nanoparticle by adjusting the ion intensity of an aqueous solution.

### Technical Solution

According to the present invention, the surface of a nanoparticle is covered with a ligand that enables stable dispersion of the nanoparticle and induces coupling with an explosive.

According to the present invention, a method of detecting an explosive includes detecting an explosive using a nanoparticle having a higher-order amine group on the surface thereof.

According to the present invention, a nanoparticle for explosive detection includes a higher-order amine group formed on the surface thereof.

According to the present invention, a method of preparing a nanoparticle for explosive detection includes binding a nanoparticle with a molecule having at least one higher-order amine group and at least one reactive group reacting with the nanoparticle.

In the present invention, the nanoparticle refers to a semiconductor nanoparticle having a diameter of less than 1000 nm. In some embodiments, the nanoparticle may have a diameter of less than 300 nm based on the definition by the National Science Foundation. In some embodiments, the nanoparticle may have a diameter of less than 100 nm based on the definition by the National Institutes of Health.

In the present invention, the nanoparticle may comprise a single nanoparticle, and many nanoparticles may be provided in the form of a single nanoparticle by aggregation. The nanoparticle may be a high-density nanoparticle, the inside of which is compact, or may be provided in the form of a nanoparticle having a partition or a space therein. In an embodiment of the present invention, the nanoparticle may be provided in the form of a monolayer or a multilayer.

In the present invention, the nanoparticle may be made of various materials, such as metal, nonmetal, ceramic, plastic, polymer, a bio substance, a semiconductor, quantum dots, or a complex material, or may be a fluorescent particle. For example, the complex material may be a particle configured such that a core made of a nonmetal material such as ceramic or polymer is coated with a metal.

According to the present invention, the nanoparticle is not theoretically limited, but the molecule formed on the surface of the nanoparticle enables the dispersion force of the nanoparticle to be maintained in a wide pH range and at high salt concentration and the independent size thereof to be maintained.

In the present invention, the higher-order amine group is a secondary or more amine group, and preferably a tertiary amine group or a quaternary amine group.

In an embodiment of the present invention, the tertiary amine group may be represented by —NR₃⁺ wherein Rs are the same as or different from each other and are each hydrogen or C1-C8 alkyl. The quaternary amine group is an ammonium group.

In the present invention, the nanoparticle having the higher-order amine group formed on the surface thereof is dispersed or dissolved in a solution, preferably an aqueous solution, thus detecting explosives in water.

In the present invention, coupling between the explosive and the nanoparticle may be enhanced by increasing the ion content in the solution, and such an increase in the ion content may result from dissolving NaCl.

In the present invention, the nanoparticle having the higher-order amine group formed on the surface thereof may be a nanoparticle configured such that a molecule having a higher-order amine group is bound to the surface of the nanoparticle.

In the present invention, the molecule may be a monomer, an oligomer such as a dimer or trimer, or a polymer compound. Preferably, the length of the molecule is shorter than the outer diameter of the nanoparticle, so that the molecule is not completely provided around the nanoparticle and is bound in a manner that extends outwards from the center of the particle under the dispersed condition, whereby the part to be coupled with explosives may be distributed on the outermost surface of the nanoparticle.

In the present invention, the molecule includes, at one side thereof, an attachment domain able to be strongly bound to the surface of the nanoparticle, and at the other side thereof, a functionality domain able to be coupled with a nitroaromatic explosive while imparting dispersion force to the nanoparticle, with an intermediate connection domain between the attachment domain and the functionality domain.

In an embodiment of the present invention, the attachment domain is capable of being strongly bound to the surface of the nanoparticle, and may include a functional group that may form stable bonding with the surface of the nanoparticle, for example, a thiol group (—SH), a dithiol group, an amine group (—NH₂, —NH), a phosphonate group (—PO₃H), a phosphide group (—P), a phosphine oxide group (—P═O), a carboxyl group (—COOH), a hydroxyl group (—OH), an imidazole group, and a diole group. Preferably useful is a dithiol group having two or more bonding portions so as to form strong bonding with the surface of the nanoparticle.

In an embodiment of the present invention, the functionality domain is located at the terminal opposite the attachment domain of the surface molecule, and is positively charged to thus achieve stable dispersion of the nanoparticle in the aqueous solution. Also, this domain indicates a domain able to be coupled with a nitroaromatic explosive such as TNT, and preferably includes tertiary amine or quaternary amine.

In an embodiment of the present invention, the connection domain is provided by strong covalent bonding to connect the attachment domain and the functionality domain to each other so as to form a single molecule. It may function to introduce different functionality domains with a predetermined attachment domain, or to introduce different attachment domains to a predetermined functionality domain. Hence, this connection domain may include a variety of functional groups for connection of desired molecules. The usable connection domain may include amide bonding (—CONH-), carbon bonding (—(CH₂)ₙ—) polyethyleneglycol (—(CH₂CH₂O)ₙ—), or triazole, wherein n is preferably an integer of 1˜100 and more preferably 1˜20. In a preferred embodiment of the present invention, the connection domain includes a hydrophobic moiety such as carbon bonding (—(CH₂)ₙ—) to thus form hydrophobic bonding with a nitroaromatic compound such as TNT. Furthermore, when the ion intensity of the solution is increased, the distance between nanoparticles may decrease, thus increasing explosive detection sensitivity.

In an embodiment of the present invention, the molecule may be represented by Chemical Formula 1 below.

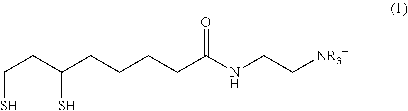

In this chemical formula, Rs are the same as or different from each other and are each hydrogen or C1-C8 alkyl.

In an embodiment of the present invention, the method of detecting an explosive may vary depending on the kind of nanoparticle, and may include changes in optical properties of the nanoparticle due to coupling between the nanoparticle and the explosive, or changes in optical properties depending on changes in the distance between nanoparticles due to coupling with explosives.

An aspect of the present invention addresses a method of preparing a nanoparticle for explosive detection, by binding a nanoparticle with a molecule having at least one higher-order amine group and at least one reactive group reacting with the nanoparticle.

In the present invention, the higher-order amine group is represented by —NR₃⁺, wherein R is hydrogen or C1-C8 alkyl, and the reactive group includes two or more functional groups selected from among a thiol group (—SH), an amine group (—NH₂, —NH), a phosphonate group (—PO₃H), a phosphide group (—P), a phosphine oxide group (—P═O), a carboxyl group (—COOH), a hydroxyl group (—OH), an imidazole group, and a diole group. The molecule is configured such that the reactive group and the higher-order amine group are connected by at least one connector selected from among amide bonding (—CONH—), carbon bonding (—(CH₂)ₙ—) wherein n is an integer of 1˜100, polyethyleneglycol (—(CH₂CH₂O)ₙ—), and triazole.

In the present invention, the nanoparticle for explosive detection may be prepared by binding the nanoparticle with a compound represented by Chemical Formula (2) below.

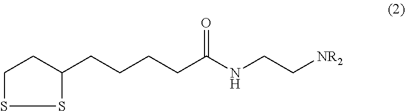

In this chemical formula, Rs are the same as or different from each other, and are each hydrogen or C1-C8 alkyl.

### Advantageous Effects

According to the present invention, a nanoparticle includes a ligand able to be coupled with an explosive while maintaining a stable dispersion state in a wide pH range and at high ion intensity. Therefore, it can be efficiently applied to development of a stable explosive sensor able to be stably coupled with an explosive despite changes in pH and ion intensity in an aqueous solution, and can thus detect explosives dissolved in sea water.

### DESCRIPTION OF DRAWINGS

FIG. 1 schematically illustrates a process of synthesizing a ligand having primary amine or tertiary amine at a terminal thereof for surface modification of a nanoparticle;

FIG. 2 schematically illustrates surface modification of a nanoparticle resulting from surface replacement of a synthesized nanoparticle in an organic solvent with a charged molecule;

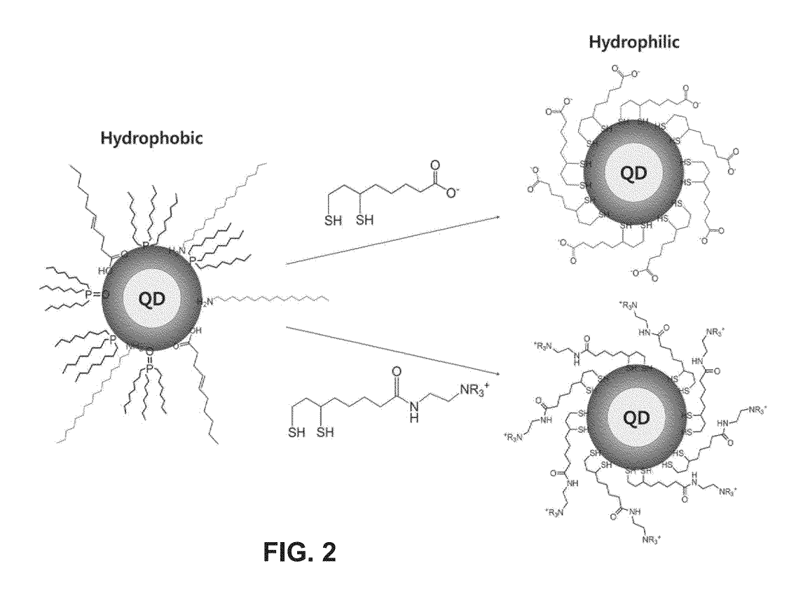

FIG. 3 illustrates absorption (left) and fluorescence (right) spectra of CdSe/CdS/ZnS (core/shell/shell) quantum dots dispersed in chloroform or water;

FIG. 4 illustrates the stability of nanoparticles in an aqueous solution depending on the type of ligand used for surface modification, including, for example, the dispersion force of a quantum dot (QD-NH₃⁻) covered with a ligand having a primary amine group at a terminal thereof and a quantum dot (QD-NH(CH₃)₂⁺) covered with a ligand having a tertiary amine group at a terminal thereof in an aqueous solution, wherein the left graph shows changes in hydrodynamic size of the quantum dots depending on changes in pH (pH 5˜10) and the right graph shows changes in hydrodynamic size of the quantum dots depending on changes in ion intensity (NaCl concentration: 0˜1.5 M), in which the quantum dots may become unstable in proportion to an increase in the hydrodynamic size and thus may not be uniformly dispersed in the aqueous solution but may agglomerate, therefore lowering the applicability of quantum dots;

FIG. 5 illustrates a graph showing changes in fluorescence of quantum dots when adding TNT to the negatively charged quantum dot (QD-COO⁻) covered with a ligand having a carboxyl group at a terminal thereof and the positively charged quantum dot (QD-NH(CH₃)₂⁺) covered with a ligand having a tertiary amine group at a terminal thereof; and

FIG. 6 illustrates a graph showing changes in fluorescence of quantum dots when adding NaCl aqueous solutions having different concentrations to the TNT-containing solution of the quantum dot (QD-NH(CH₃)₂⁺) covered with a ligand having a tertiary amine group at a terminal thereof, wherein the fluorescence intensity at each point is corrected with a quantum dot solution containing only a NaCl aqueous solution without TNT.

### MODE FOR INVENTION

### EXAMPLE 1

### Synthesis of CdSe/CdS/ZnS (Core/Shell/Shell) Quantum Dots

Synthesis of quantum dots disclosed herein is merely illustrative and is not construed as limiting the present invention.

For quantum dots having high fluorescence efficiency, CdSe quantum dots were synthesized via high-temperature pyrolysis in an organic solvent and then covered with CdS/ZnS shells, thus synthesizing quantum dots having a structure of CdSe/CdS/ZnS (core/shell/shell).

Specifically, cadmium selenide (CdSe) quantum dots were synthesized via modification of the method reported by Yu and Peng (W. W. Yu and X. Peng. Angew. Chem. Int. Edit. 2002, 41, 2368-2371). In a septum vial, 0.75 g (2.4 mmol) of cadmium acetate and 1.8 mL (6.0 mmol) of oleic acid were placed and dissolved at 100° C. in a vacuum. The completely dissolved cadmium acetate was cooled to room temperature, and then mixed with a solution of 0.47 g of selenium in 6 mL of trioctylphosphine (TOP). 15 mL of octadecene and 4 mL (12 mmol) of oleylamine were placed in a 50 mL 3-neck round-bottom flask, and then heated to 315° C. in the presence of nitrogen gas. At a raised temperature, the mixture of cadmium and selenium was rapidly injected into a reactor. After 30 sec, the heating mantle was removed, and the reaction solution was cooled to room temperature. The synthesized cadmium selenide quantum dots were diluted with hexane, and nanocrystals were precipitated with an excess of methanol using a centrifuge to remove the organic reaction residue.

Synthesis of CdSe/CdS/ZnS (core/shell/shell) quantum dots by sequentially coating the synthesized CdSe quantum dots with CdS/ZnS shells was performed with reference to the method reported by Dabbousi et al. (B. O. Dabbousi et al., J. Phys. Chem. B 1997, 101, 9463 475). In a 50 mL 3-neck round-bottom flask, 15 mL of octadecene was placed, and a CdSe solution (1.70×10⁻⁴ mmol) dispersed in 2 mL of hexane was injected at 60° C. in the presence of nitrogen gas. The hexane was removed in a vacuum. The temperature was adjusted to 120° C., and a Cd/S precursor containing 5 mL of TOP and 24.7 μL of bis(trimethylsilyl)sulfide was added using a syringe pump to a solution of 38 mg of cadmium acetate in 95 μL of oleic acid, and stirred for 30 min. The temperature was adjusted to 140° C., and a Zn/S precursor containing 44.8 μL of diethylzinc and 82.1 μL of bis(trimethylsilyl)sulfide dissolved in 10 mL of TOP was added using a syringe pump and stirred for 30 min. After completion of the reaction, the CdSe/CdS/ZnS (core/shell/shell) quantum dots were precipitated with methanol as in the CdSe quantum dots.

### EXAMPLE 2

### Synthesis of Ligand on Nanoparticle Surface with Terminal Amine

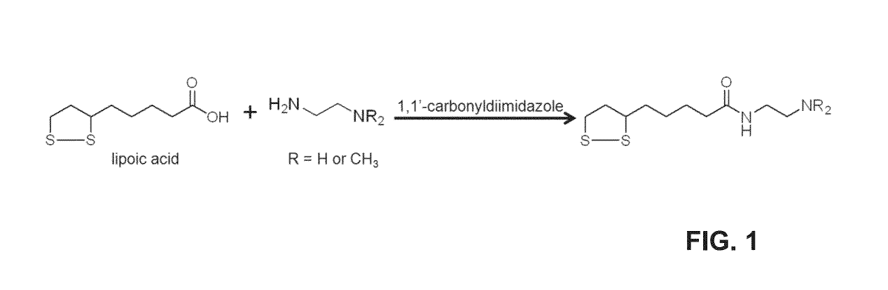

A ligand on the quantum dot surface was synthesized by binding (±)-α-lipoic acid with N,N-dimethylethylendiamine. The synthesis procedure is schematically shown in FIG. 1. Then, (±)-α-lipoic acid (20 mmol) and 1,1′-carbonyldiimidazole (26 mmol) were dissolved in 30 mL of anhydrous chloroform and stirred at room temperature for 20 min in the presence of nitrogen gas. This solution was added droplets into a flask containing N,N-dimethylethylendiamine (100 mmol) in an ice bath in the presence of nitrogen gas, and stirred for 2 hr. The product (LA-N(CH₃)₂) was washed three times with a 10% NaCl aqueous solution (80 mL) and two times with a 10 mM NaOH aqueous solution (80 mL), and then dewatered with magnesium sulfate.

### EXAMPLE 3

### Surface Modification of Quantum Dots

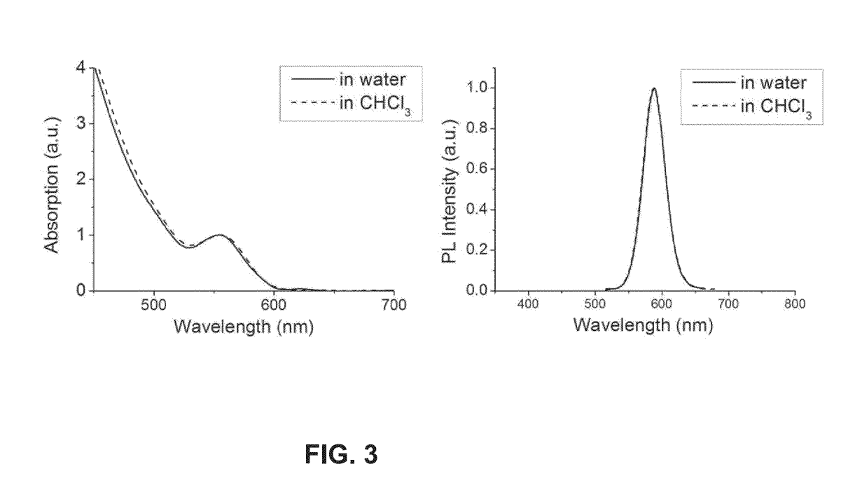

The surface of the CdSe/CdS/ZnS quantum dots synthesized in Example 1 was modified with the LA-N(CH₃)₂ ligand synthesized in Example 2. LA-N(CH₃)₂ (0.1 mmol) was dispersed in 2 mL of chloroform, and then dispersed in 2 mL of water with the addition of an aqueous solution about pH 4. The aqueous solution containing dispersed LA-N(CH₃)₂ was added with NaBH₄ (0.2 mmol), so that disulfide bonding of LA-N(CH₃)₂ was reduced, thus forming dihydrolipoic acid-tertiary amine (DHLA-N(CH₃)₂). The pH value was raised to about 10, and DHLA-N(CH₃)₂ was dispersed in chloroform, added with CdSe/CdS/ZnS quantum dots (1 nmol) dispersed in chloroform, and stirred at 60° C. for about 3 hr in the presence of nitrogen gas. The pH value was lowered to about 5, and the surface-modified quantum dots were dispersed in the aqueous solution and dialyzed using a 50,000 centrifugal filter, thus removing the surplus ligand. Based on the absorption and fluorescence spectrum results before and after surface modification of quantum dots as illustrated in FIG. 3, the surface-modified quantum dots had no changes in optical properties thereof.

### EXAMPLE 4

### Evaluation of Stability of Quantum Dots in Aqueous Solution

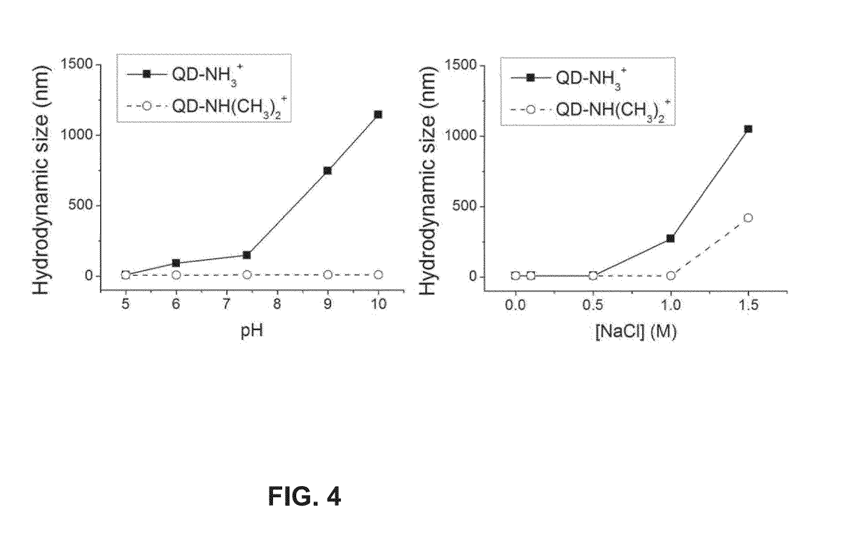

In order to evaluate stability of the quantum dots covered with the ligand having terminal tertiary amine in a wide pH range and at high ionic intensity, quantum dots covered with a ligand having terminal primary amine, conventionally useful for explosive detection, were used as a control. Changes in the hydrodynamic size of these quantum dots in the solution at different pH values and at different ionic intensities were measured. The results are graphed in FIG. 4.

### EXAMPLE 5

### Measurement of Quenching Effect of Quantum Dots by Explosive

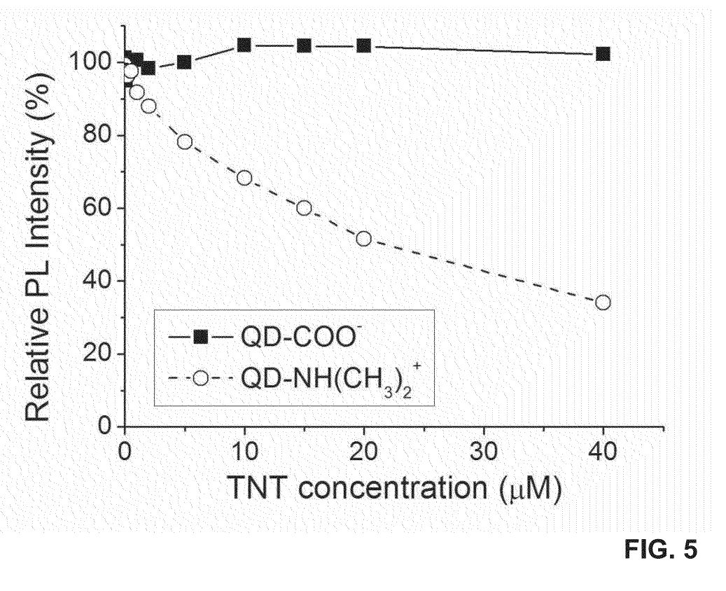

To evaluate the quenching effect of quantum dots by a nitroaromatic explosive, TNT was used. A 100 nM aqueous solution of the quantum dots of Example 3 and a 200 μM TNT aqueous solution were prepared, and used by being diluted with water upon testing. The quantum dot solution was added with TNT and excited at 400 nm, and fluorescence thereof was measured. 10 min after the addition of TNT, fluorescence was measured. The test results are shown in FIG. 5. The fluorescence of DHLA-N(CH₃)₂ surface quantum dots were decreased within 10 min by the addition of TNT. To evaluate electrostatic attraction between TNT and the amine group on the quantum dot surface, when TNT was added to the negatively charged quantum dots comprising DHLA, instead of DHLA-N(CH₃)₂, covered with a ligand having a terminal carboxyl group, fluorescence was constant regardless of the concentration of TNT.

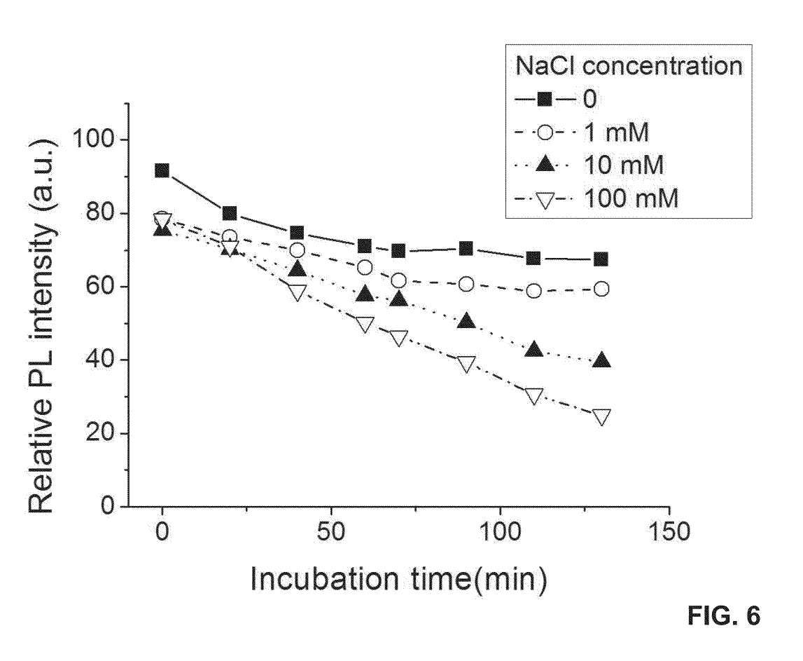

Changes in fluorescence of the mixture of quantum dots and TNT were measured with the addition of a NaCl aqueous solution. The results are shown in FIG. 6. Specifically, 100 nM quantum dots were added with 10 μM TNT, and after 30 min, sequentially added with 1 mM, 10 mM, and 100 mM NaCl aqueous solutions. Taking into consideration the effect of ionic intensity on fluorescence of the quantum dots, a solution composed exclusively of quantum dots without TNT was added with NaCl and the fluorescence thereof was corrected. When the mixed solution of quantum dots and TNT was added with the NaCl aqueous solution, the fluorescence of the quantum dots was decreased compared to when only quantum dots and TNT were provided. Also, the fluorescence was remarkably decreased in proportion to an increase in the concentration of the NaCl aqueous solution over time. This is considered to be due to the enhancement of bonding force between TNT and the ligand on the quantum dot surface at increased ion concentration. Therefore, upon appropriate control of the ligand on the quantum dot surface and the ion concentration, TNT detection efficiency in the aqueous solution can be assumed to increase.

1. A method of detecting an explosive using a nanoparticle having a higher-order amine group on a surface thereof.

2. The method of claim 1, wherein the higher-order amine group is a tertiary or more amine group.

3. The method of claim 1, wherein the explosive is detected using changes in optical properties of the nanoparticle.

4. The method of claim 1, wherein the nanoparticle is a fluorescent nanoparticle.

5. The method of claim 1, wherein the nanoparticle is a quantum dot.

6. The method of claim 1, wherein an ion concentration is increased.

7. The method of claim 6, wherein the ion concentration is increased by dissolving NaCl.

8. The method of claim 1, wherein the higher-order amine group is —NR₃⁻ in which R is hydrogen or C1-C8 alkyl.

9. The method of claim 1, wherein the nanoparticle having the higher-order amine group is formed by reacting a) a nanoparticle and b) a molecule having at least one higher-order amine group and at least one reactive group reacting with the nanoparticle.

10. The method of claim 9, wherein the reactive group is a dithiol group.

11. A nanoparticle for detecting an explosive, having a higher-order amine group on a surface thereof.

12. The nanoparticle of claim 11, wherein the nanoparticle is configured such that a nanoparticle is bound with a molecule having at least one higher-order amine group and at least one reactive group reacting with the nanoparticle.

13. The nanoparticle of claim 12, wherein the higher-order amine group is —NR₃⁺ in which R is hydrogen or C1-C8 alkyl.

14. The nanoparticle of claim 12, wherein the reactive group includes two or more functional groups selected from the group consisting of a thiol group (—SH), an amine group (—NH₂, —NH), a phosphonate group (—PO₃H), a phosphide group (—P), a phosphine oxide group (—P═O), a carboxyl group (—COOH), a hydroxyl group (—OH), an imidazole group, and a diole group.

15. The nanoparticle of claim 12, wherein the molecule is configured such that the reactive group and the higher-order amine group are connected by at least one connector selected from among amide bonding (—CONH—), carbon bonding (—(CH₂)ₙ—) wherein n is an integer of 1˜100, polyethyleneglycol (—(CH₂CH₂O)ₙ—) and triazole.

16. The nanoparticle of claim 12, wherein the molecule is represented by Chemical Formula (1) below: [chemical structure]
